(19) United States
(12) Patent Application Publication
(10) Pub. No.: US 2021/0393317 A1
(43) Pub. Date: Dec. 23, 2021

(54) VESSEL SEALING AND DISSECTION WITH CONTROLLED GAP

(71) Applicant: COVIDIEN LP, Mansfield, MA (US)

(72) Inventors: Kai Liu, Shanghai (CN); Jiewu Cao, Shanghai (CN)

(21) Appl. No.: 16/637,371

(22) PCT Filed: Aug. 7, 2017
(86) PCT No.: PCT/CN2017/096188

§ 371 Date: Feb. 7, 2020

(51) Int. Cl. A61B 18/14; A61B 90/00

(52) U.S. Cl. CPC A61B 18/1442; A61B 2018/0063; A61B 90/03

(57) ABSTRACT

A forceps includes one or more shafts having first and second jaw members at the distal end portions thereof. The jaw members are moveable from a position wherein the jaw members are spaced relative to one another to one or more approximated positions wherein the jaw members cooperate to grasp and/or seal tissue. A stop member is disposed on the first jaw member and complements a corresponding step feature defined within the second jaw member. The jaw members are tip biased at their distal surfaces such that when the jaw members move from the spaced position to a first approximated position, the distal surfaces cooperate to grasp tissue. The jaw members are further movable from the first approximated position to a second approximated position wherein the stop member bottoms out within the step feature and biases the distal surfaces to form a gap therebetween for sealing purposes.

### BACKGROUND

### Background of Related Art

The present disclosure relates to energy-based surgical instruments and, more particularly, to energy-based surgical forceps configured for dissection and treating tissue.

### Technical Field

A forceps or hemostat is a plier-like instrument which relies on mechanical action between its jaws to grasp, clamp, and constrict tissue. Energy-based forceps utilize both mechanical clamping action and energy, e.g., electrosurgical energy, ultrasonic energy, light energy, microwave energy, heat, etc., to affect hemostasis by heating tissue to coagulate and/or cauterize tissue. Certain surgical procedures require more than simply cauterizing tissue and rely on the unique combination of clamping pressure, precise energy control, and gap distance (i.e., distance between opposing jaw members when closed about tissue) to “seal” tissue. Other surgical procedures require blunt or fine dissection and typically require a uniquely shaped jaw profile to facilitate the same, e.g., tapered jaws, fine tips, tight tolerances when approximated to reduce the ingress of tissue and fluids into and between jaws. As a result, sealing tissues with these instruments is impracticable and unreliable.

As a result, surgical procedures that necessitate both tissue sealing and dissection will require the use of two or more instruments swapped into and out of the surgical cavity during the course of a given procedure. This typically adds unnecessary cost and time to the surgical procedure.

### SUMMARY

The present disclosure is directed to reusable or reposable energy-based surgical instruments, e.g., forceps, having movable, opposed jaw members that are configured for grasping, dissecting and sealing tissue.

In accordance with aspects of the present disclosure, an electrosurgical instrument, e.g., forceps, includes one or more shafts having an end effector attached at a distal end thereof. The end effector includes first and second jaw members. At least one of the jaw members is moveable about a pivot relative to the other jaw member from a position wherein the first and second jaw members are spaced relative to one another to one or more approximated positions wherein the first and second jaw members cooperate to grasp or seal tissue. Each jaw member includes a distal tissue engaging surface and a proximal tissue engaging surface.

A stop member is disposed on the first jaw member and extends therefrom. The stop member is configured to complement a corresponding step feature defined within the second jaw member. The first and second jaw members are tip biased at the distal tissue engaging surfaces such that when the first and second jaw members move from the spaced position to a first approximated position, the distal tissue engaging surfaces of the first and second jaw members cooperate to engage tissue for grasping or dissection. The first and second jaw members are further movable from the first approximated position to a second approximated position wherein the stop member bottoms out within the step feature and biases the distal tissue engaging surfaces of the first and second jaw members to form a gap therebetween for sealing purposes.

In aspects of the present disclosure, one or both of the distal tissue engaging surfaces of the jaw members includes a mechanical feature configured to facilitate dissection or grasping. The mechanical feature may be selected from a group consisting of ridges, teeth, tapering, bulbous tips, and/or contoured outer peripheral surfaces.

In aspects, when the forceps is disposed in the first approximated position, the opposing distal tissue engaging surfaces of the first and second jaw members contact one another at least at the respective distal ends thereof. In other aspects, when the forceps is disposed in the first approximated position, the opposing proximal tissue engaging surfaces of the first and second jaw members include a gap defined therebetween.

In other aspects according to the present disclosure, when the forceps is disposed in the first approximated position, the stop member is configured to partially engage the step feature. Still in other aspects, one or both of the first and second jaw members is adapted to couple to an energy source for sealing tissue.

In yet other aspects according to the present disclosure, the forceps further includes first and second shaft members which each support a corresponding one of the first and second jaw members at a respective distal end thereof and a ratchet is disposed on one of the first and second shaft members that is configured to maintain the jaw members in the second approximated position under a sealing pressure. In aspects, the ratchet maintains the sealing pressure between first and second jaw members within the range of about 3 kg/cm² to about 15 kg/cm². In still other aspects, the other of the first and second shaft members includes a complementary mechanical interface that cooperates with the ratchet to maintain the first and second jaw members in the second approximated position.

The stop member is configured to create a first gap between distal tissue engaging surfaces and a second gap between proximal tissue engaging surfaces when the first and second jaw members are disposed in the second approximated position. The first gap between the distal tissue engaging surfaces when the first and second jaw members are disposed in the second approximated position may be within the range of about 0.001 inches to about 0.006 inches. The second gap may also fall within this range. The first gap and the second gap may be the same or in aspects may be different.

In yet other aspects according to the present disclosure, the forceps further includes a pair of first and second shaft members, each of the first and second shaft members supporting a respective first and second jaw member at a distal end portion thereof. A switch is disposed on one of the first and second shaft members and a contact is disposed on the other of the first and second shaft members. The switch and the contact are adapted to connect to an energy source and cooperate to energize one or both of the first and second jaw members upon engagement thereof. In still other aspects, movement of the first and second shaft members relative to one another correspondingly moves the first and second jaw members relative to one another from the spaced apart position to the first and second approximated positions and engagement of the switch and the contact occurs after the first and second shaft members are squeezed and the first and second jaw members are moved beyond the second approximated position.

The present disclosure also relates to a forceps including a first shaft member and a second shaft member, each of the first and second shaft members having a jaw member disposed at a distal end portion thereof. The first and second shaft members are moveable relative to one another to correspondingly move the first and second jaw members about a pivot from a position wherein the first and second jaw members are spaced relative to one another to one or more approximated positions wherein the first and second shaft jaw members cooperate to grasp or seal tissue. Each jaw member includes a distal tissue engaging surface and a proximal tissue engaging surface. The forceps also includes a stop member disposed on the first jaw member that extends therefrom. The stop member is configured to complement a corresponding step feature defined within the second jaw member.

The first and second jaw members are tip biased at the distal tissue engaging surfaces such that when the first and second jaw members are moved from the spaced position to a first approximated position, the distal tissue engaging surfaces of the first and second jaw members cooperate to engage tissue for grasping or dissection. The first and second jaw members are further movable from the first approximated position to a second approximated position wherein the stop member bottoms out within the step feature and biases the distal tissue engaging surfaces of the first and second jaw members to form a gap therebetween for sealing purposes.

The present disclosure also relates to a forceps including one or more shafts having an end effector attached at a distal end thereof. The end effector including first and second jaw members, wherein one of the jaw members is moveable about a pivot relative to the other jaw member from a position wherein the first and second jaw members are spaced relative to one another to one or more approximated positions wherein the first and second jaw members cooperate to grasp or seal tissue. Each jaw member includes a distal tissue engaging surface and a proximal tissue engaging surface.

The first and second jaw members are tip biased at the distal tissue engaging surfaces such that when the first and second jaw members move from the spaced position to a first approximated position, the distal tissue engaging surfaces of the first and second jaw members cooperate to engage tissue for grasping or dissection. The first and second jaw members are further movable from the first approximated position to a second approximated position wherein a proximal portion of the distal tissue engaging surface of the first jaw member engages a proximal portion of the distal tissue engaging surface of the second jaw member and biases the distal tissue engaging surfaces of the first and second jaw members to form a gap therebetween for sealing purposes.

In aspect according to the present disclosure, one (or both) of the distal tissue engaging surfaces of the jaw members includes a mechanical feature to facilitate dissection or grasping. The mechanical feature is selected from a group consisting of ridges, teeth, tapering, bulbous tips, and contoured outer peripheral surfaces.

In other aspects according to the present disclosure, when the forceps is disposed in the first approximated position, the opposing distal tissue engaging surfaces of the first and second jaw members contact one another at least at the respective distal ends thereof. In yet other aspects, when the forceps is disposed in the first approximated position, the opposing proximal tissue engaging surfaces of the first and second jaw members include a gap defined therebetween.

In still other aspects of the present disclosure, the forceps includes first and second shaft members that support a corresponding one of the first and second jaw members at a respective distal end portion thereof. A ratchet is disposed on one of the first and second shaft members and is configured to maintain the jaw members in the second approximated position under a sealing pressure. In aspects, the ratchet maintains the sealing pressure between first and second jaw members within the range of about 3 kg/cm² to about 15 kg/cm². In aspects, the other of the first and second shaft members includes a complementary mechanical interface that cooperates with the ratchet to maintain the first and second jaw members in the second approximated position. Yet still in other aspects according to the present disclosure, the gap between the distal tissue engaging surfaces when the first and second jaw members are disposed in the second approximated position is within the range of about 0.001 inches to about 0.006 inches.

In aspects according the present disclosure, the forceps includes a pair of first and second shaft members, each of the first and second shaft members supporting a respective first and second jaw member at a distal end portion thereof. A switch is disposed on one of the first and second shaft members and a contact is disposed on the other of the first and second shaft members, the switch and the contact cooperating to energize at least one of the first and second jaw members upon engagement thereof. In aspects, movement of the first and second shaft members relative to one another correspondingly moves the first and second jaw members relative to one another from the spaced apart position to the first and second approximated positions and wherein engagement of the switch and the contact occurs after the first and second shaft members are squeezed and the first and second jaw members are moved beyond the second approximated position

### BRIEF DESCRIPTION OF THE DRAWINGS

Various aspects and features of the present disclosure are described herein with reference to the drawings wherein corresponding reference characters indicate corresponding parts throughout the drawings, and wherein:

FIG. 1A is a side view of an open electrosurgical forceps in accordance with an embodiment of the present disclosure showing a pair of opposing jaw members in a first approximated position for dissection purposes;

FIG. 1B is an enlarged view of the area of detail of FIG. 1A;

FIG. 2A is a side view of the open electrosurgical forceps of FIG. 1A showing the pair of opposing jaw members in a second approximated position for sealing purposes;

FIG. 2B is an enlarged view of the area of detail of FIG. 2A showing a gap disposed between jaw surfaces created by a stop member of one embodiment according to the present disclosure;

FIG. 2C is an enlarged view of the area of detail of FIG. 2A showing a gap disposed between jaw surfaces created by the bottoming out of a heel portion of a distal surface of one of the jaw members according to another embodiment of the present disclosure; and

FIG. 3 is a schematic illustration of a robotic surgical system configured for use in accordance with the present disclosure

### DETAILED DESCRIPTION

In this disclosure, the term “proximal” refers to a portion of a structure closer to an operator, while the term “distal” refers to a portion of the same structure further from the operator. As used herein, the term “subject” refers to a human patient or animal. The term “operator” refers to a doctor (e.g., a surgeon), a nurse, and other clinicians or care providers, and may include support personnel. The terms “generally,” “substantially,” and “about” shall be understood as words of approximation that take into account relatively little variation in the modified term(s).

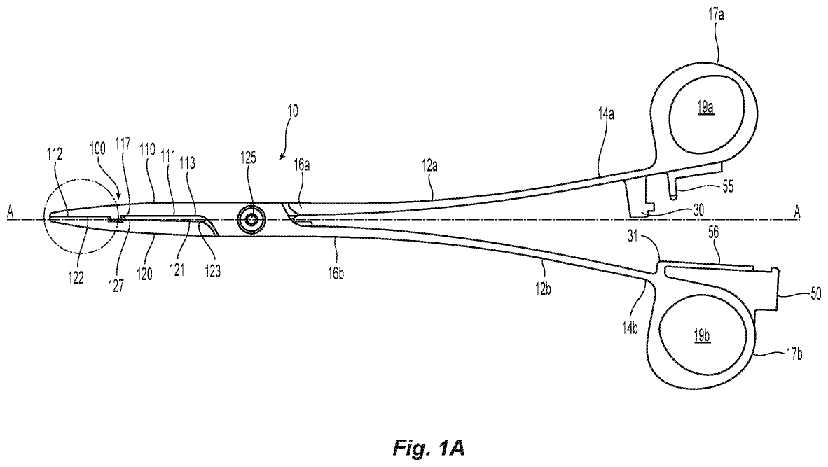

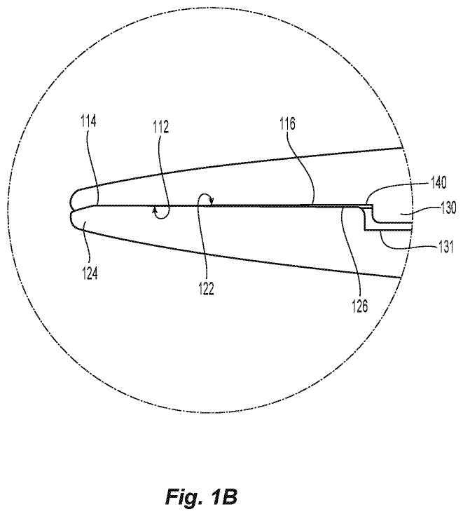

Referring now to FIG. 1, an electrosurgical instrument 10 in accordance with an embodiment of the present disclosure is configured for grasping, electrically sealing, and mechanically dissecting tissue and/or vessels in open and/or laparoscopic surgical procedures. Although only depicted herein as an open electrosurgical forceps, the concepts envisioned herein may equally apply to a laparoscopic instrument as well although the various electrical and mechanical parameters may vary.

The forceps 10 includes a first elongated shaft member 12a pivotably coupled to a second elongated shaft member 12b. The first elongated shaft member 12a includes a proximal end portion 14a and a distal end portion 16a and the second elongated shaft member 12b includes proximal end portion 14b and a distal end portion 16b. The proximal end portions 14a, 14b of the first and second shaft members 12a and 12b include first and second handle members 17a and 17b, respectively. The first and second handle members 17a and 17b are configured to allow an operator to effect movement of one or both of the first and second shaft members 12a and 12b relative to the other. More specifically, the first and second handle members 17a and 17b each define a finger hole 19a and 19b, respectively, therethrough configured to receive a finger of an operator. Finger holes 19a and 19b facilitate movement of the first and second handle members 17a and 17b relative to each other. The first and second handle members 17a and 17b, in some embodiments, are each monolithically formed with its respective shaft member 12a and 12b. Alternatively, the first and second handle members 17a and 17b may each be engaged with its respective shaft member 12a and 12b in any suitable configuration, e.g., via mechanical engagement, molding, adhesion, etc.

As shown in FIG. 1A, the first and second shaft members 12a and 12b intersect about a pivot, e.g., pivot pin 125 that is configured to both couple the first and second shaft members 12a and 12b to one another and allow rotational movement therebetween. Pivot pin 125 may be of unitary construction or part of an assembly depending upon a particular purpose. Pivot pin 125 may be insulated to electrically insulate jaw member 110 from jaw member 120.

The distal end portions 16a and 16b of the first and second shaft members 12a and 12b cooperate to define an end effector assembly 100 having opposed first and second jaw members 110 and 120. The first and second jaw members 110 and 120 extend distally from the distal end portions 16b and 16a of second and first shaft members 12b and 12a, respectively and intersect at pivot pin 125. Each jaw member 110 and 120 includes a proximal tissue engaging surface 111 and 121 respectively, and a distal tissue engaging surface 112 and 122, respectively. Proximal and distal tissue engaging surfaces 111, 121 and 112, 122 are generally disposed along the same plane relative to a longitudinal axis “A’ defined between jaw members 110 and 120 (See FIG. 1A).

Either or both the proximal and distal tissue engaging surfaces 111, 121 and 112, 122 may be made from a conductive material such that when the forceps 10 is energized, the surfaces 111, 121 and/or 112, 122 conduct energy therebetween to treat tissue in either a bipolar fashion or a monopole fashion depending upon a particular purpose. In this instance, one or more of the surfaces 111, 121 and/or 112, 122 is conductive. In embodiments, various other energy modalities may be employed to treat or seal tissue, e.g., light energy, ultrasonic energy, microwave energy, etc. In this instance, the surfaces 111, 121 and/or 112, 122 are not necessarily conductive.

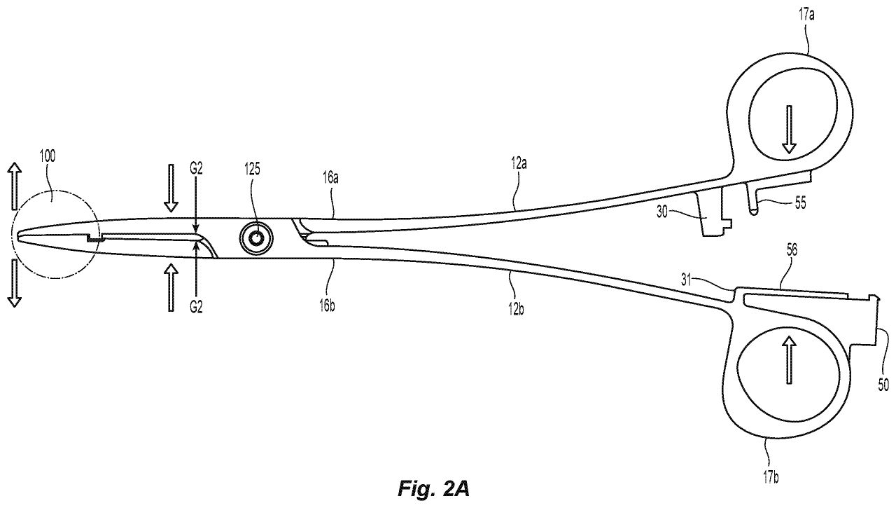

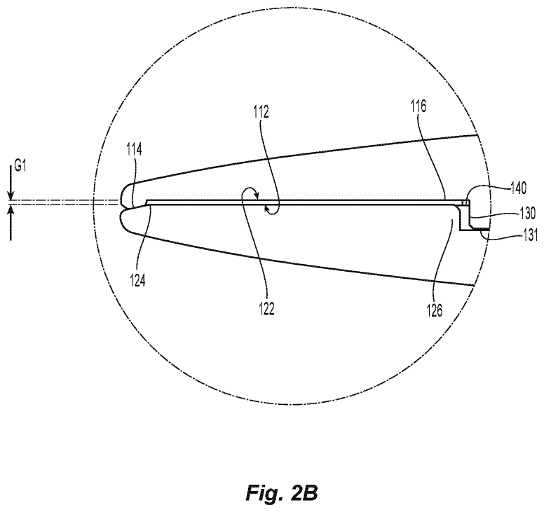

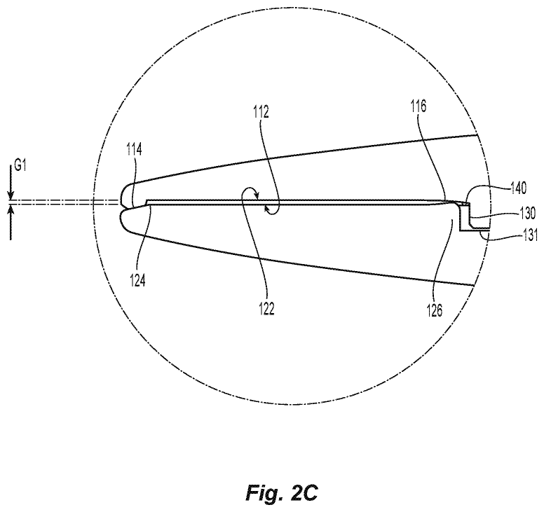

The distal engaging surfaces 112 and 122 of each jaw member 110 and 120 include proximal portions 116 and 126 and distal portions 114 and 124. The distal portions 112 and 122 of the distal tissue surfaces may be configured to facilitate grasping and dissection and include various mechanical features such as ridges, teeth, tapering, and/or a bulbous profile (see FIG. 2B). Other features are also envisioned to facilitate dissection, tissue grasping or sealing such as tapered tips, teeth, contoured outer peripheral surfaces, etc.

Each proximal tissue engaging surface 111 and 121 includes a heel portion 113 and 123 and a distal portion 117 and 127. The distal portion 117 of jaw member 110 includes a stop member 130 that is configured to be received within a complementary step feature 131 defined within jaw member 120. Stop member 103 and step feature 131 cooperate to define one or more gap distances between jaw members 110 and 120 during the sealing process as explained in more detail below. Stop member 130 may be disposed more towards the distal end of jaw member 110 in general vertical registration with step feature 131 such that, when jaw members 110 and 120 are approximated, stop member 130 is received within the step feature 131.

As best show in FIGS. 1A and 1B, in order to approximate the forceps 10 for grasping or dissection of tissue, a user orients his/her fingers into finger holes 19a and 19b of handles 17a and 17b, respectively, and moves the handles 17a and 17b towards one another which, in turn, causes the jaw members 110 and 120 to rotate around pivot pin 125 towards one another. Jaw members 110 and 120 are configured to be tip-biased such that the distal portions 114 and 124 of the distal tissue engaging portions 112 and 122 of each jaw member 110 and 120 contact one another prior to the proximal portions 116 and 126 of the distal tissue engaging portions 112 and 122. This facilitates fine grasping and dissection of tissue.

As best seen in FIG. 1B, during dissection of tissue, the proximal tissue engaging surfaces 111 and 121 are spaced relative to one another and the stop member 130 is not completely seated within the complementary step feature 131. The surgeon may open and close the jaw members 110 and 120 as needed to grasp and dissect tissue.

As best seen in FIGS. 2A and 2B, in order to effect a tissue seal, the surgeon again places his/her fingers into the finger holes 19a and 19b or handles 17a and 17b, respectively, and moves the handles 17a and 17b towards one another as indicated by directional arrow “I”. By applying additional closure pressure to handles 17a and 17b in the direction of arrow “1”, the jaw members 110 and 120 move closer to one another under the additional pressure and stop member 130 seats within the step feature 131 (See FIG. 2B). Additional pressure in the direction “I” causes the jaw members 110 and 120 to move to a second approximated position (closer together) such that the proximal tissue engaging surfaces 111 and 121 move in the direction of arrow “II” and the distal tissue engaging surfaces 112 and 122 are biased in the direction of arrow “III”. More specifically, as the jaw members 110 and 120 close under the additional pressure from the handles 17a and 17b in the direction “I”, the stop member 130 bottoms out within the step feature 131 causing the distal tissue engaging surfaces 112 and 122 to bias to a slightly open position defining a gap “G1” therebetween (See FIG. 2B). As the stop 130 bottoms out, a gap “G2” is also defined between proximal tissue engaging surfaces 111 and 121 which may be the same or different than the gap “G1” defined between distal tissue engaging surfaces 112 and 122. In embodiments, both gaps “G1” or “G2” between the opposing distal or proximal tissue engaging surfaces (112, 122 or 111, 121) are within the range of about 0.001 to about 0.006 inches. In embodiments, the proximal gap “G2” may be greater than the above range if the proximal tissue engaging surfaces 111 and 121 are not configured to facilitate tissue sealing.

As best shown in FIGS. 1A and 2A, forceps 10 also includes a distal connector 50 that is configured to connect to an energy source, e.g., electrosurgical generator (not shown), optical generator (not shown), ultrasonic generator (not shown) or other known energy or heat source (not shown). Forceps 10 may also include a ratchet 30 disposed on shaft 12a that is configured to engage shaft 12b or a complementary mechanical interface 31 disposed on shaft 12b. Any complementary mechanical mating features known in the art are envisioned that will accomplish this purpose. An electro-mechanical switch 55 may be disposed on one of the shafts, e.g., shaft 12a, that is configured to engage a contact 56 disposed on the opposite shaft, e.g., shaft 12b. When the handles 17a and 17b are fully approximated and the jaw members 110 and 120 are engaged about tissue, the switch 55 is configured to engage the contact 56 and energize the jaw members 110 and 120. In embodiments, the ratchet 30 may be configured to maintain the jaw members 110 and 120 in an approximated position under a sealing pressure to facilitate sealing. Typically the ratchet 30 maintains the first and second jaw members 110 and 120 in the second approximated position appropriate for sealing tissue. Additional movement of the handles 17a and 17b beyond the ratcheted position (with the jaw members 110 and 120 in the second approximated position) will engage the switch 55 with the contact 56 to energize the tissue.

When the ratchet 30 is engaged with the jaw members 110 and 120 positioned about tissue, the handles 17a and 17b may be released and the ratchet 30 will maintain the jaw members 110 and 120 about tissue under a sealing pressure. In embodiments, the sealing pressure is within a range of about 3 kg/cm² to about 15 kg/cm².

Typically, the switch 55 is not actuated when the ratchet 30 is in an engaged position. Upon addition movement of the handles 17a and 17b in the direction of arrow “I”, the switch 55 moves into engagement with contact 56 and the jaw members 110 and 120 are energized to treat and seal tissue. In embodiments, the additional movement of the handles 17a and 17b in the direction of arrow “I” does not necessarily apply more pressure to the jaw members 110 and 120 since the stop member 130 is already bottomed out within the step feature 131 at this point. In other embodiments, the forceps 10 may be designed to apply a last amount of pressure to assure that the pressure falls within the sealing pressure range (as specified above) just prior to activation of the switch 55 or when moving the handles 17a and 17b in the direction of arrow “I” from the ratcheted position to the switch activation position.

When sealing is complete and the electrosurgical energy source (not shown) is shut off, the first and second handle members 17a and 17b may be returned to the open position to release tissue held between the first and second jaw members 110 and 120. Tissue may be regrasped for further treatment or other tissue may be dissected, grasped or sealed.

FIG. 2C shows another embodiment of the forceps 10 according to the present disclosure that differs slightly from the embodiment of FIG. 2B. As such, only those features that vary from the forceps 10 shown in FIG. 2B will be described. Moreover, user operation of the forceps 10 is identical to the forceps 10 described in FIGS. 2A and 2B, however, the structure of jaw members 110 and 120 differs slightly. More particularly, jaw members 110 and 120 include similar distal tissue engaging surfaces 112 and 122, however, the proximal portions 116 and 126 of the distal tissue engaging surfaces 112 and 122 are designed to bottom out upon engagement of the ratchet 30 and ratchet interface 31 to bias the distal tissue engaging surfaces 112 and 122 apart from one another and form gap G1 between surfaces.

As the proximal portions 116 and 126 bottom out, the gap “G2” may also be defined between proximal tissue engaging surfaces 111 and 121 which may be the same or different than the gap “G1” defined between distal tissue engaging surfaces 112 and 122. Also, in this instance, the stop member 130 does not necessarily bottom out within step feature 131. Similar to the forceps 10 described above with respect to FIG. 2B, in embodiments, both gaps “G1” or “G2” between the opposing distal or proximal tissue engaging surfaces (112, 122 or 111, 121) may be within the range of about 0.001 to about 0.006 inches. In other embodiments, the proximal gap “G2” may be greater than the above range if the proximal tissue engaging surfaces 111 and 121 are not configured to facilitate tissue sealing.

The embodiments disclosed herein may also be configured to work with robotic surgical systems and what is commonly referred to as “Telesurgery.” Such systems employ various robotic elements to assist the operator and allow remote operation (or partial remote operation) of surgical instrumentation. Various robotic arms, gears, cams, pulleys, electric and mechanical motors, etc. may be employed for this purpose and may be designed with a robotic surgical system to assist the operator during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep a subject (e.g., a patient) for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

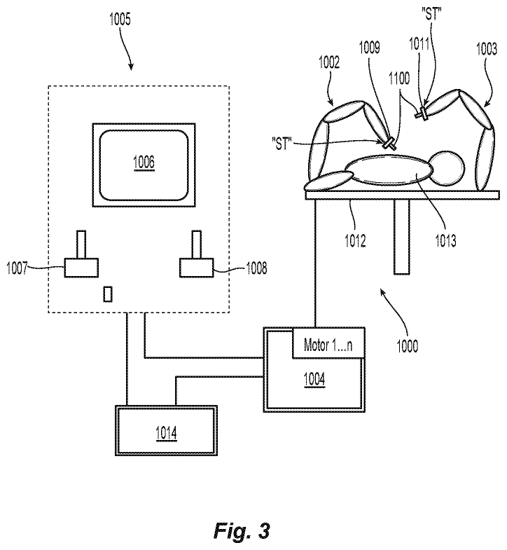

As shown in FIG. 3, a medical work station is shown generally as work station 1000 and generally may include a plurality of robot arms 1002 and 1003; a control device 1004; and an operating console 1005 coupled with control device 1004. Operating console 1005 may include a display device 1006, which may be set up in particular to display three-dimensional images; and manual input devices 1007 and 1008, by means of which an operator (not shown), for example a surgeon, may be able to telemanipulate robot arms 1002 and 1003 in a first operating mode.

Each of the robot arms 1002 and 1003 may include a plurality of members, which are connected through joints, and an attaching device 1009 and 1011, to which may be attached, for example, a surgical tool “ST” supporting an end effector 1100, in accordance with any one of several embodiments disclosed herein, as will be described in greater detail below.

Robot arms 1002 and 1003 may be driven by electric drives (not shown) that are connected to control device 1004. Control device 1004 (e.g., a computer) may be set up to activate the drives, in particular by means of a computer program, in such a way that robot arms 1002 and 1003, their attaching devices 1009 and 1011 and thus the surgical tool (including end effector 1100) execute a desired movement according to a movement defined by means of manual input devices 1007 and 1008. Control device 1004 may also be set up in such a way that it regulates the movement of robot arms 1002 and 1003, and/or of the drives.

Medical work station 1000 may be configured for use on a patient 1013 lying on a patient table 1012 to be treated in a minimally invasive manner by means of end effector 1100. Medical work station 1000 may also include more than two robot arms 1002 and 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to control device 1004, in which are stored, for example, pre-operative data from patient/living being 1013 and/or anatomical atlases.

While several embodiments of the disclosure have been shown in the drawings and described herein, it is not intended that the disclosure be limited thereto, as it is intended that the disclosure be as broad in scope as the art will allow and that the specification be read likewise. For example, although only shown as an open forceps, it is contemplated that the jaw members 110 and 120 and the various aforedescribed features relating thereto may be utilized on an endoscopic forceps as well. Therefore, the above description should not be construed as limiting, but merely as examples of particular embodiments. Those skilled in the art will envision other modifications within the scope and spirit of the claims appended hereto.

What is claimed is:

1. A forceps, comprising:
at least one shaft having an end effector attached at a distal end thereof, the end effector including first and second jaw members, at least one jaw member moveable about a pivot relative to the other jaw member from a position wherein the first and second jaw members are spaced relative to one another to one or more approximated positions wherein the first and second jaw members cooperate to grasp or seal tissue, each jaw member including a distal tissue engaging surface and a proximal tissue engaging surface; and
a stop member disposed on the first jaw member and extending therefrom, the stop member configured to complement a corresponding step feature defined within the second jaw member,
wherein the first and second jaw members are tip biased at the distal tissue engaging surfaces such that when the first and second jaw members move from the spaced position to a first approximated position, the distal tissue engaging surfaces of the first and second jaw members cooperate to engage tissue for grasping or dissection, and
wherein when the first and second jaw members are further moved from the first approximated position to a second approximated position, the stop member bottoms out within the step feature and biases the distal tissue engaging surfaces of the first and second jaw members to form a gap therebetween for sealing purposes.

2. The forceps according to claim 1 wherein at least one of the distal tissue engaging surfaces of at least one of the jaw members includes a mechanical feature to facilitate dissection or grasping.

3. The forceps according to claim 2 wherein the mechanical feature is selected from a group consisting of ridges, teeth, tapering, bulbous tips, and contoured outer peripheral surfaces.

4. The forceps according to claim 1 wherein when the forceps is disposed in the first approximated position, the opposing distal tissue engaging surfaces of the first and second jaw members contact one another at least at the respective distal ends thereof.

5. The forceps according to claim 1 wherein when the forceps is disposed in the first approximated position, the opposing proximal tissue engaging surfaces of the first and second jaw members include a gap defined therebetween.

6. The forceps according to claim 1 wherein when the forceps is disposed in the first approximated position, the stop member is configured to partially engage the step feature.

7. The forceps according to claim 1 wherein at least one of the first and second jaw members is adapted to couple to an energy source for sealing tissue.

8. The forceps according to claim 1 further comprising:
first and second shaft members, each of the first and second shaft members supports a corresponding one of the first and second jaw members at a respective distal end portion thereof; and
a ratchet disposed on one of the first and second shaft members configured to maintain the jaw members in the second approximated position under a sealing pressure.

9. The forceps according to claim 8 wherein the ratchet maintains the sealing pressure between first and second jaw members within the range of about 3 kg/cm² to about 15 kg/cm².

10. The forceps according to claim 8 wherein the other of the first and second shaft members includes a complementary mechanical interface that cooperates with the ratchet to maintain the first and second jaw members in the second approximated position.

11. The forceps according to claim 1 wherein the gap between the distal tissue engaging surfaces when the first and second jaw members are disposed in the second approximated position is within the range of about 0.001 inches to about 0.006 inches.

12. The forceps according to claim 1 wherein the stop member is configured to create a first gap between distal tissue engaging surfaces and a second gap between proximal tissue engaging surfaces when the first and second jaw members are disposed in the second approximated position.

13. The forceps according to claim 12 wherein the first gap and the second gap are different.

14. The forceps according to claim 12 wherein the first gap and the second gap are the same.

15. The forceps according to claim 1 further comprising:
a pair of first and second shaft members, each of the first and second shaft members supporting a respective first and second jaw member at a distal end portion thereof; and
a switch disposed on one of the first and second shaft members and a contact disposed on the other of the first and second shaft members, the switch and the contact cooperating to energize at least one of the first and second jaw members upon engagement thereof.

16. The forceps according to claim 15 wherein movement of the first and second shaft members relative to one another correspondingly moves the first and second jaw members relative to one another from the spaced apart position to the first and second approximated positions and wherein engagement of the switch and the contact occurs after the first and second shaft members are squeezed and the first and second jaw members are moved beyond the second approximated position.

17. A forceps, comprising:
a first shaft member and a second shaft member, each of the first and second shaft members having a jaw member disposed at a distal end portion thereof, the first and second shaft members moveable relative to one another to correspondingly move the first and second jaw members about a pivot from a position wherein the first and second jaw members are spaced relative to one another to one or more approximated positions wherein the first and second shaft jaw members cooperate to grasp or seal tissue, each jaw member including a distal tissue engaging surface and a proximal tissue engaging surface;
a stop member disposed on the first jaw member and extending therefrom, the stop member configured to complement a corresponding step feature defined within the second jaw member,
wherein the first and second jaw members are tip biased at the distal tissue engaging surfaces such that when the first and second jaw members are moved from the spaced position to a first approximated position, the distal tissue engaging surfaces of the first and second jaw members cooperate to engage tissue for grasping or dissection, and
wherein when the first and second jaw members are further moved from the first approximated position to a second approximated position, the stop member bottoms out within the step feature and biases the distal tissue engaging surfaces of the first and second jaw members to form a gap therebetween for sealing purposes.

18. The forceps according to claim 17 wherein the gap between the distal tissue engaging surfaces when the first and second jaw members are disposed in the second approximated position is within the range of about 0.001 inches to about 0.006 inches.

19. The forceps according to claim 17 wherein the stop member is configured to create a first gap between distal tissue engaging surfaces and a second gap between proximal tissue engaging surfaces when the first and second jaw members are disposed in the second approximated position.

20. The forceps according to claim 19 wherein the first gap and the second gap are different.
